(19) United States
(12) Patent Application Publication
(10) Pub. No.: US 2022/0296611 A1
(43) Pub. Date: Sep. 22, 2022

(54) PHARMACEUTICAL COMPOSITION FOR PREVENTING OR TREATING CANCER

(71) Applicant: SEOUL NATIONAL UNIVERSITY R&DB FOUNDATION, Seoul (KR)

(72) Inventor: Seong Chan YEON, Seoul (KR)

(21) Appl. No.: 17/833,095

(22) Filed: Jun. 6, 2022

Related U.S. Application Data
(63) Continuation of application No. PCT/KR2020/018271, filed on Dec. 14, 2020.

(30) Foreign Application Priority Data
Dec. 13, 2019 (KR) 10-2019-0166966
Dec. 14, 2020 (KR) 10-2020-0174443

(51) Int. Cl. A61K 31/575; A61K 31/4184; A61K 45/06

(52) U.S. Cl. CPC A61K 31/575; A61K 31/4184; A61K 45/06

(57) ABSTRACT

A composition according to an embodiment of the present disclosure includes UCDA and a benzimidazole-based compound. A method for treating cancer according to an embodiment of the present disclosure includes administering the composition to a subject in need thereof. The composition has an excellent anticancer effect and has the benefit of having less side effects as cell toxicity of the composition is lower than conventional anticancer drugs used in existing treatments.

### CROSS-REFERENCE TO RELATED APPLICATION AND CLAIM OF PRIORITY

The present application is a continuation of application to International Application No. PCT/KR2020/018271 with an international filing date of Dec. 14, 2020, which claims the benefit of Korean Patent Application Nos. 10-2019-0166966 filed on Dec. 13, 2019 and 10-2020-0174443 filed on Dec. 14, 2020 at the Korean intellectual property office, the disclosures of which are incorporated by reference herein in their entirety.

### BACKGROUND

### 1. Technical Field

The present invention relates to a composition for preventing or treating cancer.

### 2. Background Art

Despite numerous studies being conducted, cancer is a significant problem worldwide and its conquest still remains as a major challenge in medical science. Chemotherapy is one of the major methods in cancer treatment therapy, and generally refers to treatment of cancer using one or more anticancer drugs (chemotherapeutic agents).

Many anticancer drugs are characterized by suppressing mitosis, and inhibit cell proliferation by targeting rapidly dividable cancer cells. Some anticancer drugs stop cell division, and some anticancer drugs trigger apoptosis to kill cells. Apart from such traditional chemotherapy, many efforts are being made to provide a more targeted therapy.

Most of various anticancer drugs currently used for the treatment of cancer have a problem of high toxicity. Specifically, chemotherapy has lots of side effects such as renal toxicity, liver toxicity, severe nausea and vomiting, bone marrow suppression, hair loss, cytopenia and the like. Therefore, it is urgently necessary to develop a new anticancer drug with high anticancer effects and without being harmful to the human body.

### SUMMARY

It is an aspect of the present invention to provide a composition for preventing or treating cancer, which includes ursodeoxycholic acid (UDCA) and a benzimidazole-based compound.

1. A composition including ursodeoxycholic acid (UDCA) and a benzimidazole-based compound.

2. The composition according to the above 1, wherein the UDCA is at least one selected from the group consisting of UDCA, TUDCA and GUDCA.

3. The composition according to the above 1, wherein the benzimidazole-based compound is at least one selected from the group consisting of fenbendazole, albendazole, mebendazole and flubendazole.

4. The composition according to the above 1, wherein the UDCA and the benzimidazole-based compound are included in a molar ratio of 1:0.5 to 5.

5. The composition according to the above 1, wherein the UDCA is TUDCA, and the benzimidazole-based compound is fenbendazole.

6. The composition according to the 1 above, further including at least one selected from the group consisting of vitamin E tocopherol, vitamin E succinate and omega 3.

7. The composition according to the above 1, further including vitamin E succinate and omega 3.

8. The composition according to the above 7, wherein the UDCA, benzimidazole-based compound, vitamin E succinate and omega 3 are included in a molar ratio of 1:0.5 to 5:0.5 to 3:0.1 to 2.

9. The composition according to the above 1, wherein the composition may prevent or treat cancer selected from the group consisting of liver cancer, testicular cancer, glioblastoma, oral cancer, basal cell cancer, brain tumor, gallbladder cancer, biliary tract cancer, colorectal cancer, laryngeal cancer, retinocytoma, ampulla of Vater cancer, bladder cancer, peritoneal cancer, adrenal cancer, non-small cell lung cancer, tongue cancer, small cell lung cancer, small intestine cancer, meningioma, esophageal cancer, renal pelvic ureter cancer, renal cancer, malignant bone tumor, malignant soft tissue tumor, malignant pimple tumor, malignant melanoma, eye tumor, urethral cancer, gastric cancer, breast cancer, sarcoma, pharyngeal cancer, cervical cancer, endometrial cancer, uterine sarcoma, prostate cancer, metastatic brain tumor, rectal cancer, vaginal cancer, spinal cord tumor, salivary gland cancer, tonsil cancer, squamous cell cancer, lung cancer and anal cancer.

10. The composition according to the above 9, wherein the cancer is liver cancer or cervical cancer.

The present invention provides a composition for preventing or treating cancer, including ursodeoxycholic acid (UDCA) and a benzimidazole-based compound. Compared to therapeutics used as the existing anticancer drugs, the composition of the present invention has advantages of more excellent anticancer effects and less side effects.

### BRIEF DESCRIPTION OF THE DRAWINGS

FIG. 1 is a flowchart schematically illustrating MTT assay process.

FIG. 2 illustrates confirmation of anticancer effects on cervical cancer by comparing the cell viability of HeLa cells in each treatment group.

FIG. 3 illustrates confirmation of anticancer effects on liver cancer by comparing the cell viability of Hepa cells and expressing the same as a percentage in each treatment group.

FIG. 4 illustrates the collected results of FIG. 3 as a single straight line graph.

FIG. 5 illustrates confirmation of anticancer effects on liver cancer by comparing the cell viability of Hepa cells and expressing the same as a percentage in each treatment group.

FIG. 6 shows the data of the results of anticancer effects on liver cancer as numerical valued.

FIG. 7 illustrates confirmation of anticancer effects on liver cancer by comparing the cell viability of Hepa cells and expressing the same as a percentage in each treatment group.

FIG. 8 illustrates confirmation of anticancer effects on liver cancer by counting the number of living cells among Hepa cells in each treatment group.

FIG. 9 illustrates the collected results of FIG. 8 as a single straight line graph.

FIG. 10 shows the data of FIGS. 7 and 8 as numerical values

FIG. 11 illustrates confirmation of pH3 protein through Western blot analysis.

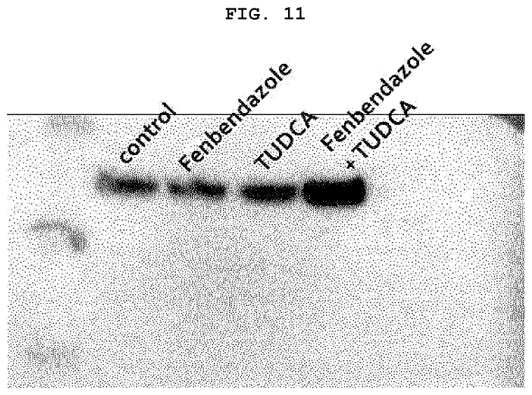

### DETAILED DESCRIPTION

Hereinafter, the present invention will be described in detail.

The present invention relates to a pharmaceutical composition for preventing or treating cancer, which includes ursodeoxycholic acid (UDCA) and a benzimidazole-based compound.

UDCA is an abbreviation of ursodeoxycholic acid, and UDCA according to the present invention includes all of UDCA, tauroursodeoxycholic acid (TUDCA) in which UDCA is conjugated with taurine, and glycoursodeoxycholic acid (GUDCA) in which UDCA is conjugated with glycine.

In the present invention, the UDCA, TUDCA or GUDCA may be a synthetic product or may be isolated and identified from the nutria gallbladder, and may include a component recombined after extraction of a water-soluble component, but it is not limited thereto.

With regard to extraction from the nutria gallbladder, specifically, it may be extracted from nutria bile, and the extraction method may use any extraction method known in the art without limitation thereof. For example, freeze-dried extraction, hot water extraction, extraction using lower alcohols having 1 to 4 carbon atoms, in the case of resin Z-802: CDCA, 99% extraction, HPLC extraction, ultrafiltration extraction, supercritical fluid chromatography extraction, capillary electrophoresis extraction method, or the like, may be used, but it is not limited thereto.

In the present invention, the benzimidazole-based compound may be, for example, fenbendazole, albendazole, mebendazole or flubendazole, and any benzimidazole-based compound used as an anthelmintic may be possible, but it is not limited thereto. The combination of UDCA and the benzimidazole-based compound may be variously selected, for example, all possible combinations of UDCA, GUDCA, TUDCA, and fenbendazole, albendazole, mebendazole and flubendazole exemplified above may be available. For example, TUDCA may be used as UDCA, and fenbendazole may be used as a benzimidazole-based compound.

A content ratio of UDCA and the benzimidazole-based compound is not particularly limited, and for example, UDCA and benzimidazole may be included in a molar ratio of 1:0.5 to 5. For example, the molar ratio may be 1:0.5 to 5, 1:0.5 to 4, 1:0.5 to 3, 1:1 to 5, 1:1 to 4, 1:1 to 3, 1:1 to 2.5, 1:1.5 to 5, 1:2 to 2.5, but it is not limited thereto. The above ratio may be appropriately adjusted depending on patients subjected to administration or results of clinical trial.

The pharmaceutical composition of the present invention may further include vitamin E tocopherol, vitamin E succinate or omega 3. In such a case, a size of tumor may be reduced to further improve anticancer efficacy.

The pharmaceutical composition of the present invention may further include at least one from the group consisting of vitamin E tocopherol, vitamin E succinate and omega 3, and for example, may further include vitamin E tocopherol, vitamin E succinate and omega 3, may further include vitamin E tocopherol and omega 3, and may further include vitamin E succinate and omega 3.

A mixing ratio of the above ingredients is not particularly limited, and for example, in a case of further including vitamin E succinate and omega, the molar ratio of UDCA, a benzimidazole-based compound, vitamin E succinate and omega 3 may be, for example, 1:0.5 to 5:0.5 to 3:0.1 to 2, 1:0.5 to 3:0.5 to 1.5:0.2 to 1.5, 1:0.8 to 2.8:0.8 to 1.2:0.4 to 1.2, but it is not limited thereto.

As the cancer to be prevented or treated in the present invention, any cancer may be applied without limitation thereof, and the cancer may include, for example, at least one selected from the group consisting of liver cancer, testicular cancer, glioblastoma, oral cancer, basal cell cancer, brain tumor, gallbladder cancer, biliary tract cancer, colorectal cancer, laryngeal cancer, retinocytoma, ampulla of Vater cancer, bladder cancer, peritoneal cancer, adrenal cancer, non-small cell lung cancer, tongue cancer, small cell lung cancer, small intestine cancer, meningioma, esophageal cancer, renal pelvic ureter cancer, renal cancer, malignant bone tumor, malignant soft tissue tumor, malignant pimple tumor, malignant melanoma, eye tumor, urethral cancer, gastric cancer, breast cancer, sarcoma, pharyngeal cancer, cervical cancer, endometrial cancer, uterine sarcoma, prostate cancer, metastatic brain tumor, rectal cancer, vaginal cancer, spinal cord tumor, salivary gland cancer, tonsil cancer, squamous cell cancer, lung cancer and anal cancer, and specifically liver cancer or cervical cancer, but it is not limited thereto.

In the present invention, the UDCA and the benzimidazole-based compound may be used by appropriately selecting a compound, a water-soluble extract, a recombinant thereof, etc. according to the desired formulation.

In the present invention, the pharmaceutical composition may further include a pharmaceutically acceptable carrier in addition to the above ingredients. The pharmaceutically acceptable carrier may include those commonly used, such as lactose, dextrose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinyl pyrrolidone, water, methyl hydroxybenzoate, propyl hydroxybenzoate, talc, magnesium stearate and mineral oil, but it is not limited thereto. Further, the pharmaceutical composition of the present invention may include diluents or excipients such as fillers, extenders, binders, wetting agents, disintegrants, surfactants, and other pharmaceutically acceptable additives. The pharmaceutically acceptable additives may be included in an amount of 0.1 to 99.9 parts by weight (“wt. parts”) based on the composition, but the amount thereof may be adjusted by clinical trials, and it is not limited thereto.

The pharmaceutical composition of the present invention may be administered orally or parenterally (e.g., application or intravenous, subcutaneous, or intraperitoneal injection), and for example, may be applied through oral administration or administration via injection, but it is not limited thereto.

UDCA, benzimidazole-based anthelmintics, etc. are used as oral agents, and the pharmaceutical composition of the present invention may be orally administered. Therefore, it is possible to administer and take the composition in a more convenient method compared to the conventional anticancer drugs that require an administration method such as intratumoral administration and vascular administration.

The pharmaceutical composition of the present invention has effects of protecting the liver because of including the UDCA-based compound, such that liver damage caused by taking the drug can be reduced while having less side effects thereof. Further, due to anticancer synergistic effects of the UDCA-based compound, the composition may exhibit superior effects than those of a pharmaceutical composition containing only the benzimidazole-based compound, and can be easily taken as an oral drug. Therefore, it is expected that the medication compliance may also be excellent.

Solid preparations for oral administration may include powders, granules, tablets, capsules, soft capsules, pills, and the like, but they are not limited thereto. Liquid formulations for oral use may include suspensions, solutions, emulsions, syrups, aerosols, etc. Other than water and liquid paraffin, which are commonly used as simple diluents, various excipients such as wetting agents, sweeteners, fragrances, and preservatives, etc. may be included, but they are not limited thereto. Preparations for parenteral administration may be formulated and used in the form of external preparations and sterile injection preparations such as sterilized aqueous solutions, solutions, non-aqueous solutions, suspensions, emulsions, eye drops, eye ointments, syrups, suppositories, aerosols, etc. according to the conventional methods. For example, the pharmaceutical composition may be produced and used in the form of creams, gels, patches, sprays, ointments, plasters, lotions, liniment agents, eye ointments, eye drops, pasta agents or cataplasma, but it is not limited thereto. A composition for topical administration may be anhydrous or aqueous depending on clinical prescription, and non-aqueous solvents and suspensions may include propylene glycol, polyethylene glycol, vegetable oils such as olive oil, and injectable esters such as ethyl oleate, but it is not limited thereto. As a base of the suppository, witepsol, macrogol, tween 61, cacao butter, laurin butter, glycerogelatin, etc. may be used, but it is not limited thereto.

A preferred dosage of the pharmaceutical composition of the present invention may vary depending on the absorption of the active ingredient in the body, the age, gender, and degree of obesity of the patient, but can be appropriately selected by those skilled in the art. However, to achieve the desired effects, in the case of oral administration, the composition of the present invention may be generally administered to an adult in an amount of 0.0001 to 500 mg/kg, and preferably, 0.001 to 500 mg/kg per kilogram of body weight per day. The composition may be administered once a day or in several divided doses. The above dosage and administration method do not limit the scope of the present invention in any way.

Hereinafter, examples will be described in detail to more concretely understand the present invention.

Experimental Method

1. Cell Number Counting Method

After culturing cells, the cells were resuspended in a fresh medium. After obtaining 200 μL of the cell suspension, 10 μL of trypsin blue with a dilution factor of 2× was added thereto.

A hemocytometer equipped with a coverslip was prepared, and each chamber was filled with the suspension, followed by counting the number of cells. A specific counting method is as follows.

1) Live cells/ml=No. of cells counted/No. of squares counted×Dilution factor×1000

2) Dead cells/ml=No. of dead cells/No. of squares counted×Dilution factor×100

3) Percentage Viability=No. of live cells/Total No. of cells×100

2. Cell Culture

After establishing a sterile environment (clean bench, autoclave, 70% Ethanol, etc.), a cell growth medium (DMEM, FBS, Glutamine, Penicillin/Streptomycin) was prepared. Cells were identified and cultured under the above conditions.

3. MTT Assay

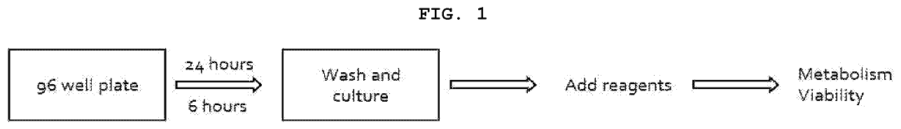

As illustrated in the flow chart shown in FIG. 1, the degree of cytotoxicity was evaluated through MTT assay.

4. Western Blot Assessment

Western blot assessment was performed to confirm a degree of production of pH3 protein in Hepa cells or HeLa cells treated with TUDCA or the like.

Total protein was obtained from the cells with a radio-immunoprecipitation assay buffer. The protein was digested in an 8 to 12% polyacrylamide gel and transferred to a nitrocellulose membrane. As a blocking buffer, 5% nonfat dry milk (1% Tween-20, in 20 mM TBS, pH 7.6) was used, and incubated with a primary antibody in the blocking buffer.

The primary antibody is an antibody obtained from Cell Signaling Technology in the USA or other countries, and a secondary antibody is anti-mouse or anti-rabbit IgG bound to HRP, and was obtained from GE Healthcare, Little Chalfont, UK.

Thereafter, the density of the band was measured by Western blot analysis.

Results of Experiment

1. Comparison of Cell Viability of HeLa Cells

(1) HeLa cells are cervical cancer cell lines. In this experiment, experimental groups were divided as follows: specifically, a group in which HeLa cells are not treated, that is, no treatment group (NT); a doxorubicin-treated group (Doxo); a fenbendazole-treated group (Fen); a TUDCA treated group (TUDCA); and a group treated with a mixture of fenbendazole and TUDCA (Fen+TUDCA), and cell viability was compared over time (24 h, 48 h) after treatment. Treatment was implemented using TUDCA at a concentration of 1000 μM, fenbendazole at a concentration of 1.2 μM, and doxorubicin at a concentration of 10 μM, respectively. Further, the mixture of fenbendazole and TUDCA was prepared by mixing both compounds with the above concentrations at the same dose. Further, all treatment groups were treated with the corresponding compounds at the same dose.

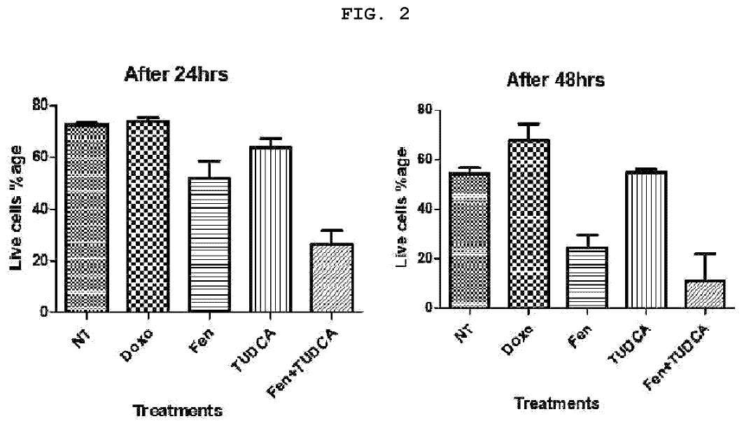

(2) As shown in FIG. 2, when compared to the effect of doxorubicin which is generally used as an anticancer drug, it could be seen that all of fenbendazole, TUDCA, the mixture of fenbendazole and TUDCA have more excellent apoptotic effects at 24 h and 48 h after treatment than doxorubicin, thereby expecting better anticancer effects. Further, in the case of the fenbendazole and TUDCA mixture treatment group, the effect was the most excellent over all times.

2. Comparison of Cell Viability of Hepa Cells

(1) In the present invention, all liver cancer cell lines were Murin hepatoma cell lines (Hepa1c1c7), hereinafter, referred to as Hepa cells. In this experiment, the following definitions were applied: no treatment group (NT); a fenbendazole treatment group (Fen); a TUDCA 200 μmol treatment group (TUDCA 200); a TUDCA 500 μmol treatment group (TUDCA 500); a TUDCA 1000 μmol treatment group (TUDCA 1000); a mixture of 1.2 μmol fenbendazole and 200 μmol TUDCA (1.2+200); a mixture of 1.2 μmol fenbendazole and 500 μmol TUDCA (1.2+500); a mixture of 1.2 μmol fenbendazole and 1000 μmol TUDCA (1.2+1000), respectively. Each treatment group was treated with the corresponding compound at the concentration set to 100 nM. A mixing ratio in the mixture was 1:1 unless otherwise specified.

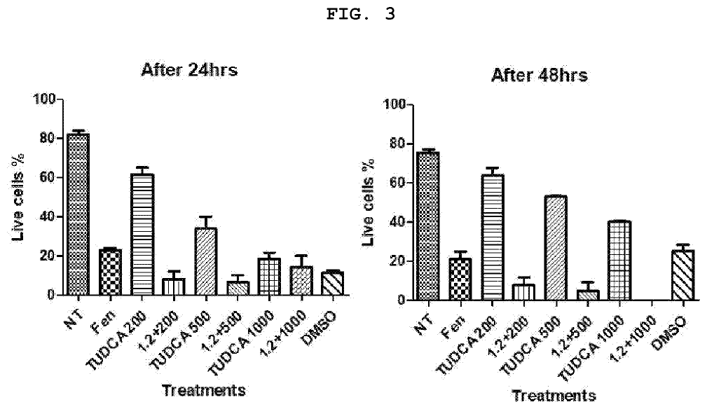

(2) As shown in FIG. 3, in the liver cancer cell lines, all treatment groups exhibited apoptosis effects compared to the NT group, and specifically, the group treated with a mixture of fenbendazole and TUDCA had the best effects. It could be seen that the higher the concentration of TUDCA, the better the apoptosis effect at 48 h, that is, the anticancer effects.

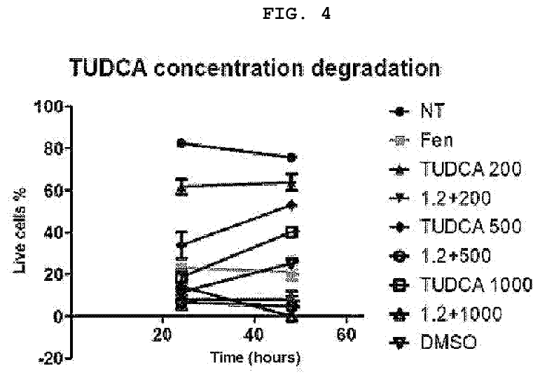

FIG. 4 illustrates the collected results of FIG. 3 as a dotted line graph, and the results are the same as shown in FIG. 3.

3. Comparison of Hepa Cell Viability in Fenbendazole and UDCA Compound Treatment Groups

(1) This experiment was performed to compare cell viability by treating liver cancer cell lines with each of the mixtures of fenbendazole and UDCA, fenbendazole and TUDCA, fenbendazole and GUDCA, respectively, while the control was treated with DMSO.

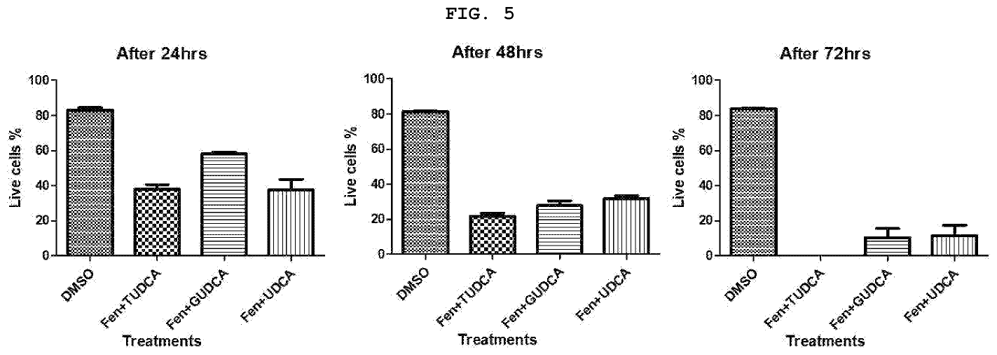

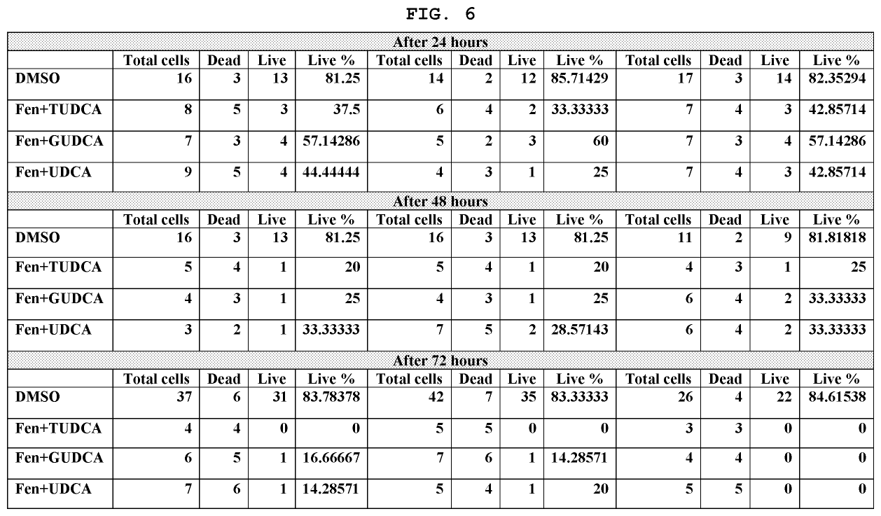

(2) As shown in FIG. 5, the apoptosis effect was excellent in all groups treated with the mixtures, and it was confirmed that the effect was the most excellent in the mixture of fenbendazole and TUDCA. Specifically, the cell viability was close to 0% at 72 h after treatment with the fenbendazole and TUDCA mixture. FIG. 6 shows the results of this experiment as numerical values, and FIG. 5 shows the average values thereof.

4. Comparison of Cell Viability According to Treatment of Various Mixtures

(1) This experiment was performed to confirm cell viability by treating liver cancer cell lines with various mixtures including cisplatin, doxorubicin, paclitaxel, and fenbendazole, which are used as conventional anticancer drugs.

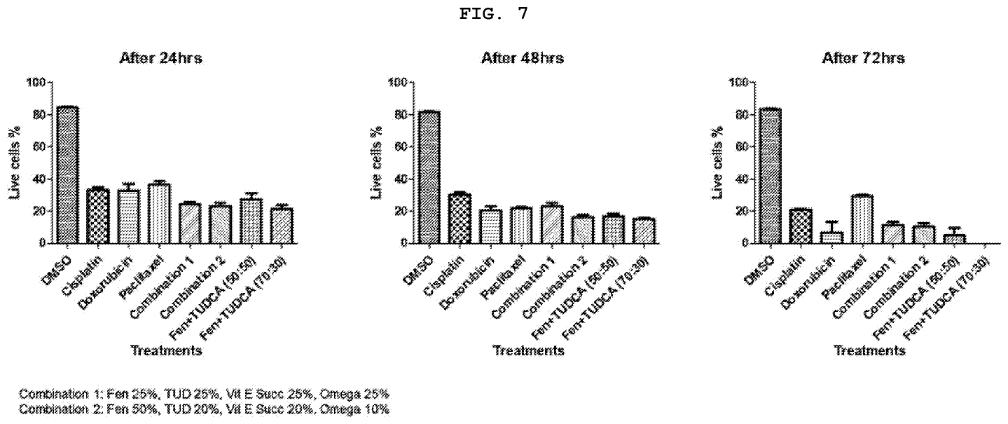

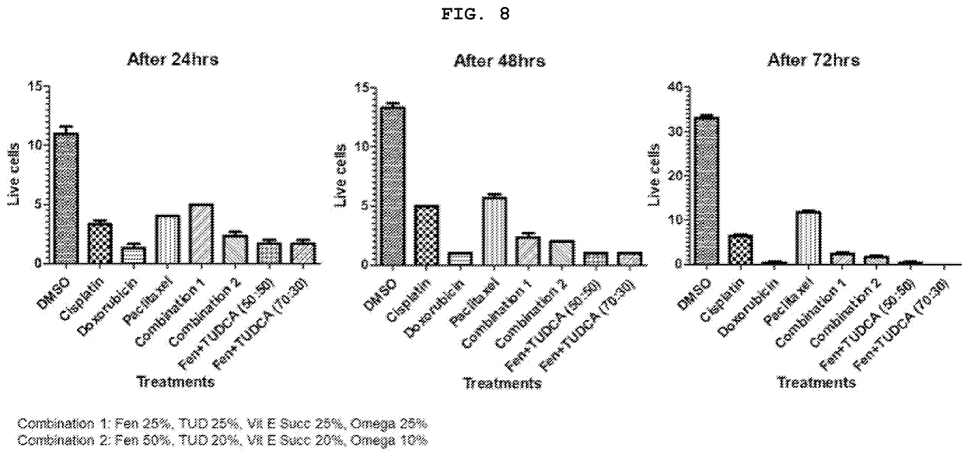

(2) As shown in FIGS. 7 and 8, it could be confirmed from the results at 24 h and 48 h after treatment that the apoptosis effect in the group treated with the mixture containing fenbendazole was similar or superior to or than the conventional anticancer drug treatment group. This means that anticancer effects of the mixture containing fenbendazole are excellent. Further, in the case of 72 h after treatment, it could be seen that the cell viability is close to 0% in the group treated with a 70:30 mixture of fenbendazole and TUDCA.

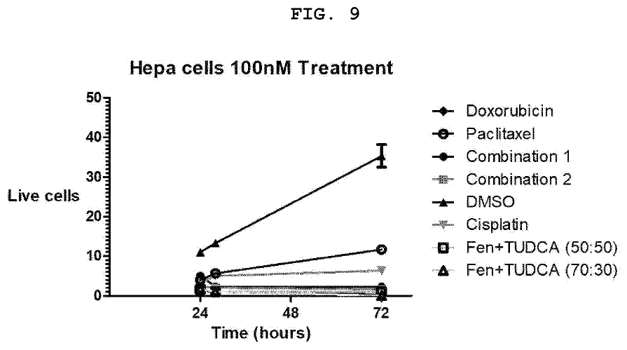

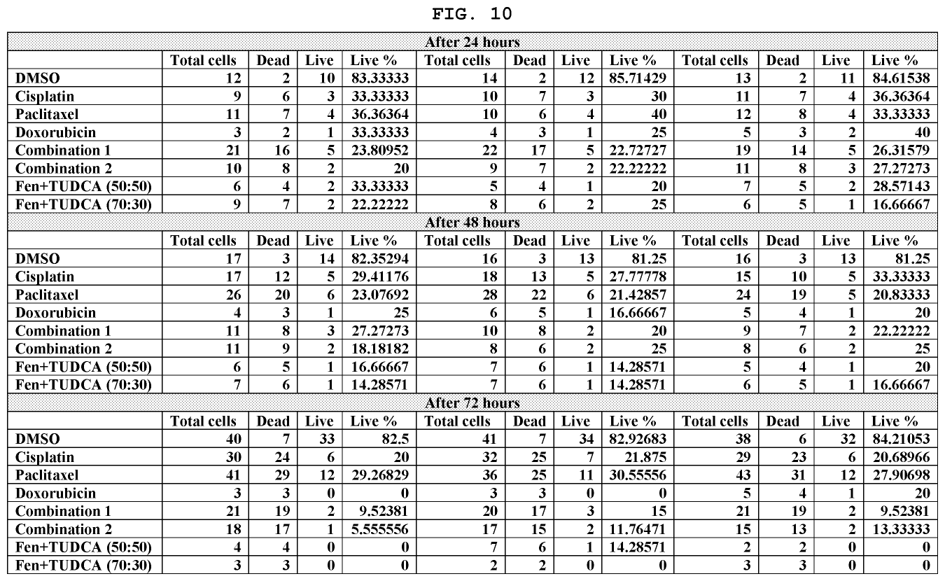

Further, it could be seen that the higher the mixing ratio of fenbendazole in the mixture of fenbendazole and TUDCA, the more excellent the anticancer effects. FIG. 9 shows the collected results of FIG. 8 as a single straight line graph, FIG. 10 shows data of FIGS. 7 and 8 as numerical values, and FIGS. 7 and 8 show the average of the three experimental values as a graph.

5. Western Blot Assessment

(1) The pH3 protein confirmed in this experiment is a protein increasing when the cell cycle is interrupted, and appearance of a distinct band due to an increase in the pH3 protein means that the cell cycle is effectively stopped.

(2) In the group treated with fenbendazole and TUDCA together, it could be seen that the pH3 protein was remarkably increased through the distinctly darkened band as a result of Weston blot, and this result means that the cell cycle of the cancer cell line was interrupted, thus indicating expression of anticancer effects. As shown in FIG. 10, it could be seen that the band was most intense and distinct in the fenbendazole and TUDCA mixture treatment group. Through this, it could be considered that the cell cycle of cancer cell lines is better interrupted when treated with the mixture of fenbendazole and TUDCA than the group treated with fenbendazole and TUDCA alone, thereby indicating better anticancer effects.

What is claimed is:

1. A composition comprising ursodeoxycholic acid (UDCA) and a benzimidazole-based compound.

2. The composition according to claim 1, wherein the UDCA is at least one selected from the group consisting of UDCA, tauroursodeoxycholic acid (TUDCA) and glycoursodeoxycholic acid (GUDCA).

3. The composition according to claim 1, wherein the benzimidazole-based compound is at least one selected from the group consisting of fenbendazole, albendazole, mebendazole and flubendazole.

4. The composition according to claim 1, wherein the UDCA and the benzimidazole-based compound are included in a molar ratio of 1:0.5 to 5.

5. The composition according to claim 1, wherein the UDCA is TUDCA, and the benzimidazole-based compound is fenbendazole.

6. The composition according to claim 1, further comprising at least one selected from the group consisting of vitamin E tocopherol, vitamin E succinate and omega-3 fatty acid.

7. The composition according claim 1, further comprising vitamin E succinate and omega-3 fatty acid.

8. The composition according to claim 7, wherein the UDCA, benzimidazole-based compound, vitamin E succinate and omega-3 fatty acid are included in a molar ratio of 1:0.5 to 5:0.5 to 3:0.1 to 2.

9. The composition according to claim 1, wherein the UDCA is at least one selected from the group consisting of UDCA, tauroursodeoxycholic acid (TUDCA) and glycoursodeoxycholic acid (GUDCA); and
the benzimidazole-based compound is at least one selected from the group consisting of fenbendazole, albendazole, mebendazole and flubendazole.

10. The composition according to claim 9, wherein the UDCA and the benzimidazole-based compound are included in a molar ratio of 1:0.5 to 5.

11. A method for treating cancer, the method comprising administering a composition comprising ursodeoxycholic acid (UDCA) and a benzimidazole-based compound to a subject in need thereof.

12. The method of claim 11, wherein the UDCA is at least one selected from the group consisting of UDCA, TUDCA and GUDCA.

13. The method of claim 11, wherein the benzimidazole-based compound is at least one selected from the group consisting of fenbendazole, albendazole, mebendazole and flubendazole.

14. The method of claim 11, wherein the UDCA and the benzimidazole-based compound are included in a molar ratio of 1:0.5 to 5.

15. The method of claim 11, wherein the UDCA is TUDCA, and the benzimidazole-based compound is fenbendazole.

16. The method of claim 11, further comprising at least one selected from the group consisting of vitamin E tocopherol, vitamin E succinate and omega-3 fatty acid.

17. The method of claim 11, further comprising vitamin E succinate and omega-3 fatty acid.

18. The method of claim 17, wherein the UDCA, benzimidazole-based compound, vitamin E succinate and omega-3 fatty acid are included in a molar ratio of 1:0.5 to 5:0.5 to 3:0.1 to 2.

19. The method of claim 11, wherein the cancer is at least one selected from the group consisting of liver cancer, testicular cancer, glioblastoma, oral cancer, basal cell cancer, brain tumor, gallbladder cancer, biliary tract cancer, colorectal cancer, laryngeal cancer, retinocytoma, ampulla of Vater cancer, bladder cancer, peritoneal cancer, adrenal cancer, non-small cell lung cancer, tongue cancer, small cell lung cancer, small intestine cancer, meningioma, esophageal cancer, renal pelvic ureter cancer, renal cancer, malignant bone tumor, malignant soft tissue tumor, malignant pimple tumor, malignant melanoma, eye tumor, urethral cancer, gastric cancer, breast cancer, sarcoma, pharyngeal cancer, cervical cancer, endometrial cancer, uterine sarcoma, prostate cancer, metastatic brain tumor, rectal cancer, vaginal cancer, spinal cord tumor, salivary gland cancer, tonsil cancer, squamous cell cancer, lung cancer and anal cancer.

20. The method of claim 11, wherein the cancer is liver cancer or cervical cancer.
